(19) United States
(12) Patent Application Publication
(10) Pub. No.: US 2004/0087034 A1
(43) Pub. Date: May 6, 2004

(54) Test strip

(76) Inventor: Ching Ho Lien, Taipei (TW)

Correspondence Address: Keith Kline, PRO-TECHTOR INTERNATIONAL SERVICES, 20775 Norada Court, Saratoga, CA, 95070-3018, US

(21) Appl. No.: 10/285,057

(22) Filed: Oct. 30, 2002

(51) Int. Cl. G01N 33/558

(52) U.S. Cl. 436/514; 210/658

(57) ABSTRACT

A test strip is constructed to include a substrate, a first sample-collection pad adhered to one side of the substrate, the first sample-collection pad having a side notch forming with the substrate a blood sample receiving chamber, and a second sample-collection pad adhered to the outer surface of the first sample-collection pad in parallel to the substrate and defining a blood sample entrance accessible to the blood sample receiving chamber.

### BACKGROUND OF THE INVENTION

1. Field of the Invention

The present invention relates to test strips for medical examination and, more particularly, to such a test strip, which can absorb blood sample from different directions.

2. Description of the Related Art

Various portable medical examination apparatus are commercially available for examining a particular item or multiple items of blood substances with test strips. With respect to blood sample collection, regular test strips for use with medical examination apparatus include two types, i.e., the direct blood collection type and the indirect blood collection type. A test strip of direct blood collection type must be attached to the punched point of the patient's finger to collect the blood. When a test strip of indirect blood collection type is used, the user or examiner drops blood sample to the test strip with an implement. Because the amount of blood sample applied to the test strip affect the examination result. The examiner must carefully drop the blood sample to the test strip.

### SUMMARY OF THE INVENTION

The present invention has been accomplished under the circumstances in view. It is the main object of the present invention to provide a test strip, which can collect blood sample from the top as well as from the side. According to one embodiment of the present invention, the test strip comprises a substrate, a first sample-collection pad adhered to one side of the substrate, the first sample-collection pad having a side notch forming with the substrate a blood sample receiving chamber, and a second sample-collection pad adhered to the outer surface of the first sample-collection pad in parallel to the substrate and defining a blood sample entrance accessible to the blood sample receiving chamber. In another embodiment of the present invention, the second sample-collection pad is formed of two symmetrical halves arranged in parallel with a narrow gap left therebetween in communication with the blood sample receiving chamber.

### BRIEF DESCRIPTION OF THE DRAWINGS

FIG. 1 is an exploded view of a test strip according to the first embodiment of the present invention.

FIG. 2 is an elevational assembly view of the test strip according to the first embodiment of the present invention.

FIG. 3 is a side view of the test strip according to the first embodiment of the present invention.

FIG. 4 is a top view of the test strip according to the first embodiment of the present invention.

FIG. 5 is an exploded view of a test strip according to the second embodiment of the present invention.

FIG. 6 is an elevational assembly view of the test strip according to the second embodiment of the present invention.

FIG. 7 is an exploded view of a test strip according to the third embodiment of the present invention.

FIG. 8 is an elevational assembly view of the test strip according to the third embodiment of the present invention.

FIG. 9 is an exploded view of a test strip according to the fourth embodiment of the present invention.

FIG. 10 is an elevational assembly view of the test strip according to the fourth embodiment of the present invention.

FIG. 11 is an exploded view of a test strip according to the fifth embodiment of the present invention.

FIG. 12 is an elevational assembly view of the test strip according to the fifth embodiment of the present invention.

### DETAILED DESCRIPTION OF THE PREFERRED EMBODIMENT

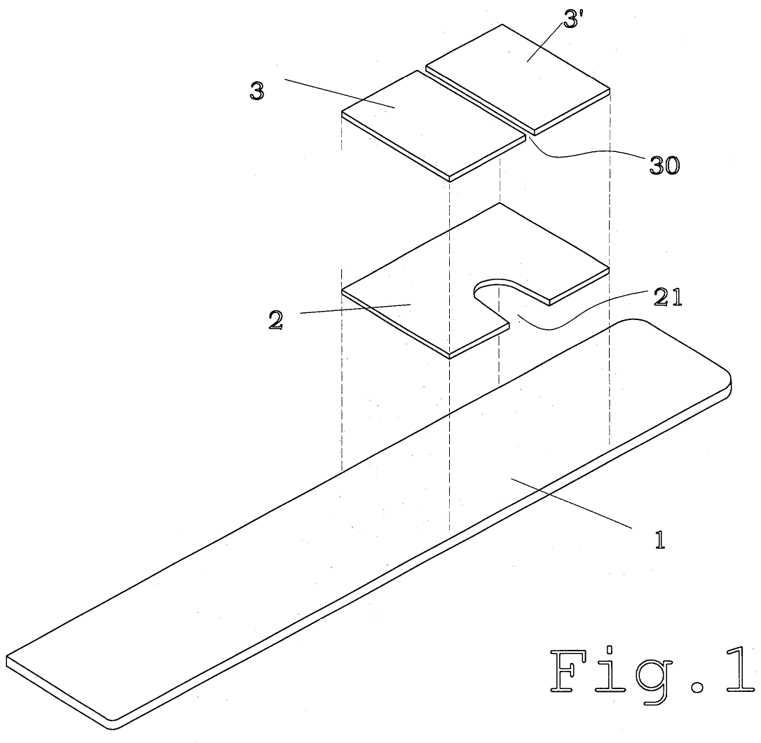

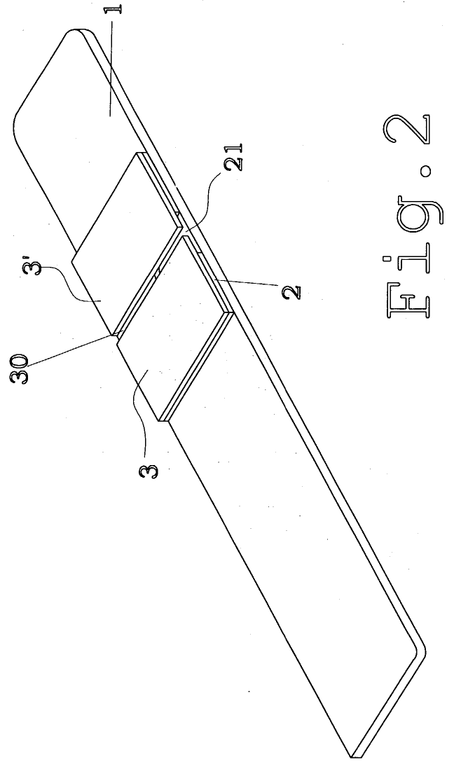

Referring to FIGS.1 and 2, a test strip is shown comprising a narrow, elongated strip of substrate 1, a first sample-collection pad 2, and at least one, for example, two second sample-collection pads 3 and 3′. The first sample-collection pad 2 is adhered to one side of the substrate 1. The second sample-collection pads 3 and 3′ are adhered to the outer surface of the first sample-collection pad 2 and arranged in parallel to the substrate 1. The first sample-collection pad 2 has a side notch 21. Preferably, the side notch 21 has a smoothly arched profile. When the first sample-collection pad 2 adhered to the substrate 1, the side notch 21 defines with the substrate 1 a blood sample receiving chamber. When the second sample-collection pads 3 and 3′ adhered to the first sample-collection pad 2, a narrow gap 30 is left between the second sample-collection pads 3 and 3′ in communication with the side notch 21 of the first sample-collection pad 2.

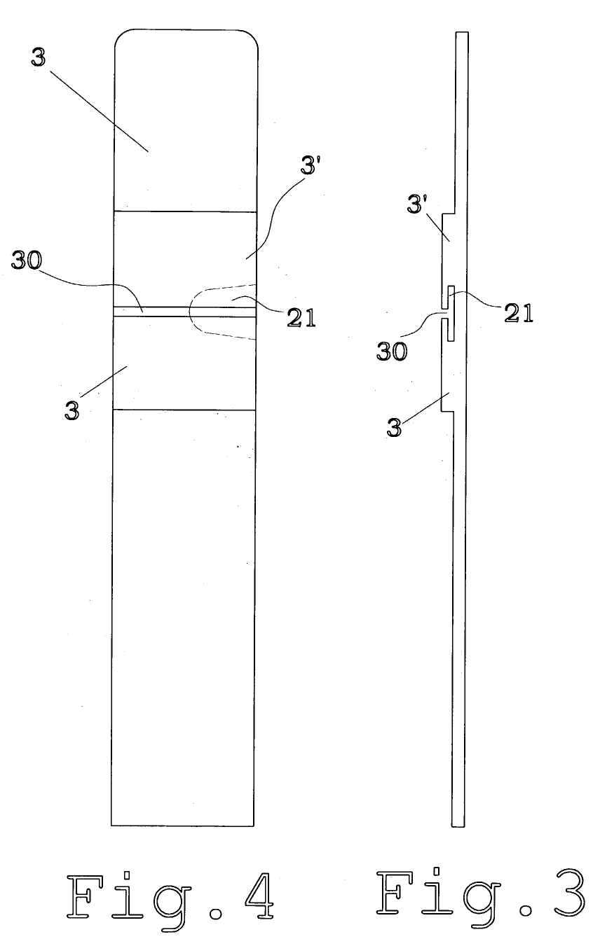

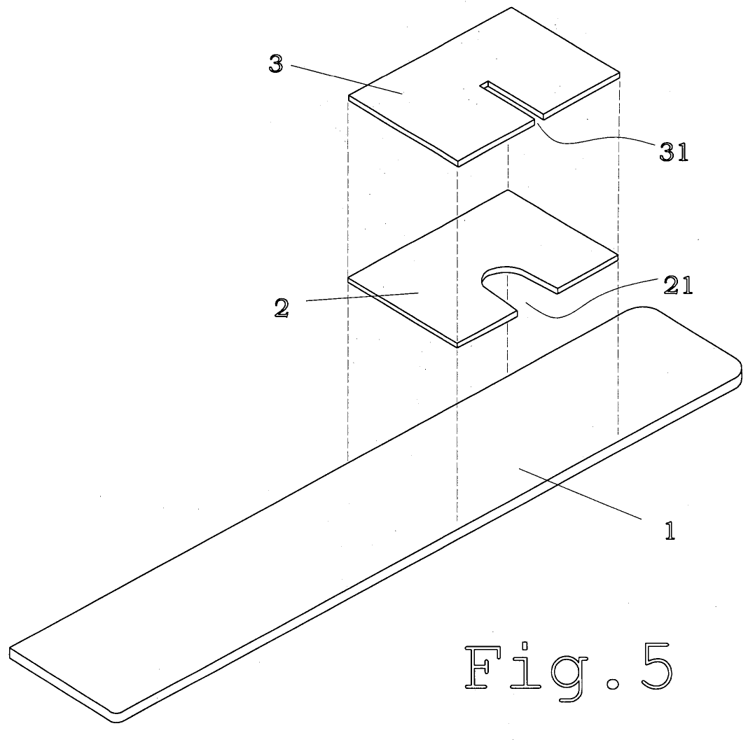

Referring to FIGS. 3 and 4, when the test strip inserted into the test apparatus and the reagent permeated into the sample-collection pads 2, 3, and 3′, the test strip is attached to the punched point of the patient's finger with the side notch 21 of the first sample-collection pad 2 approached the blood flowing out of the punched point of the patient finger, enabling the sample blood to be guided into the receiving chamber defined by the substrate 1 and the first sample-collection pad 2 and then absorbed by the sample-collection pads 2, 3, and 3′ to make a chemical reaction with the reagent in the sample-collection pads 2, 3, and 3′, for enabling the test apparatus to read the value of the assigned substance in the test sample (blood). In another test method, the user or examiner can drop the test sample (patient's blood or serum) into the gap 30 between the second sample-collection pads 3 and 3′, enabling the test sample to be absorbed by the sample-collection pads 2, 3, and 3′ for further examination.

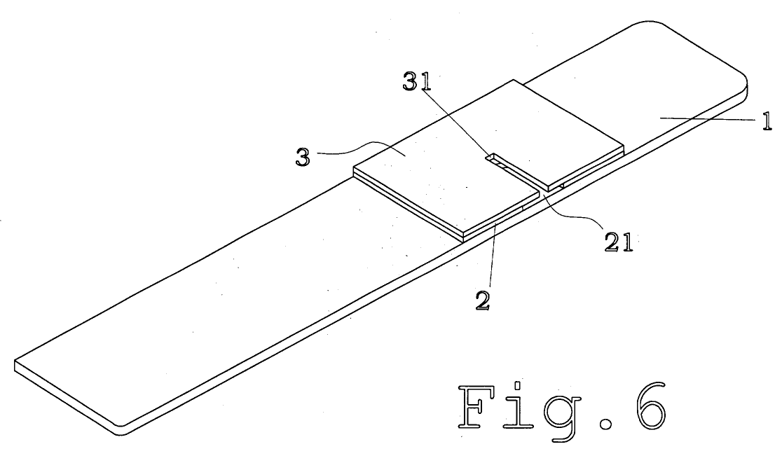

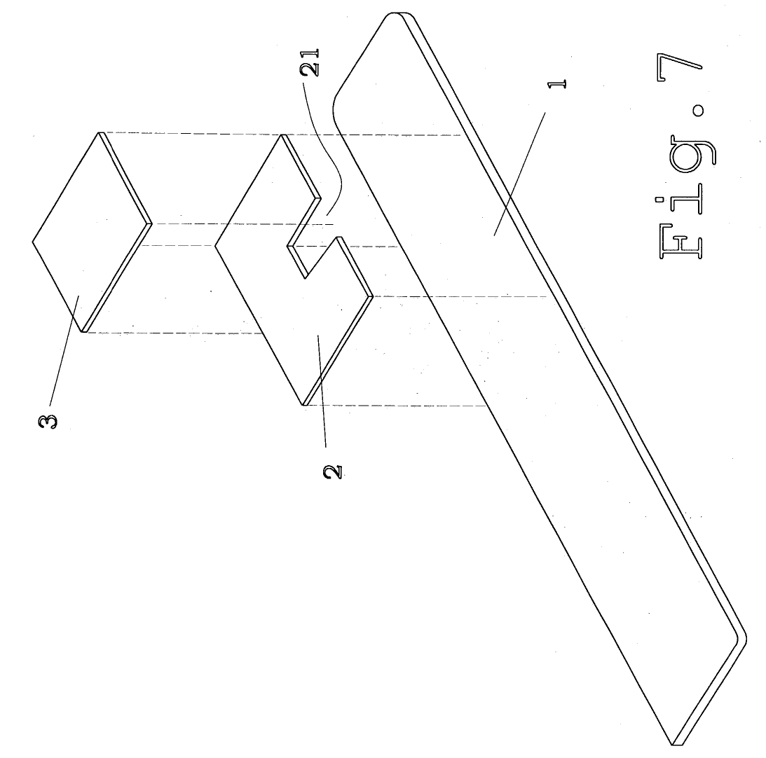

FIGS. 5 and 6 show a test strip according to the second embodiment of the present invention. According to this embodiment, the test strip is comprised of a substrate 1, a first sample-collection pad 2 adhered to one side of the substrate, and a second sample-collection pad 3 adhered to the outer surface of the first sample-collection pad 2. The first sample-collection pad 2 has a side notch 21, forming a blood sample receiving chamber on the substrate 1. The second sample-collection pad 3 and the first sample-collection pad 2 have the same size. The second sample-collection pad 3 has a narrow transverse slot 31 extended to one side in communication with the side notch 21. This embodiment achieves the same effect as the aforesaid first embodiment. Same as the test strip of the aforesaid first embodiment, the test strip of the second embodiment can be inserted into a test apparatus to absorb the reagent and then to collect the blood sample from the patient. Alternatively, the test strip can absorb the test sample (the patient's blood) at first and then inserted into the examination apparatus to examine the value of the assigned substance in the patient blood.

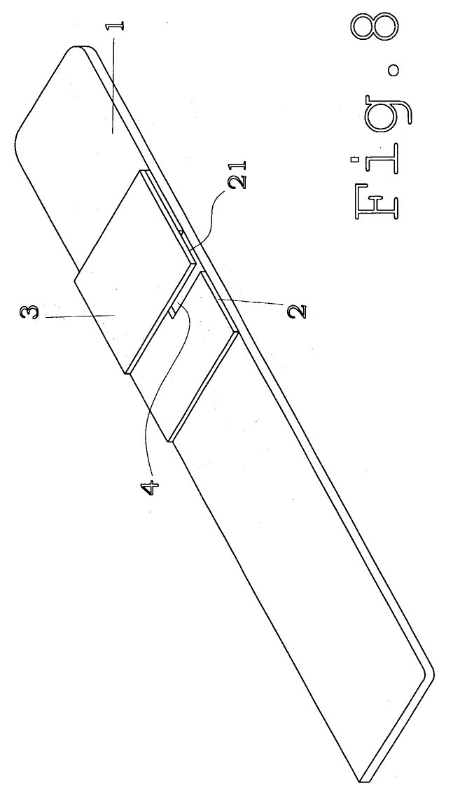

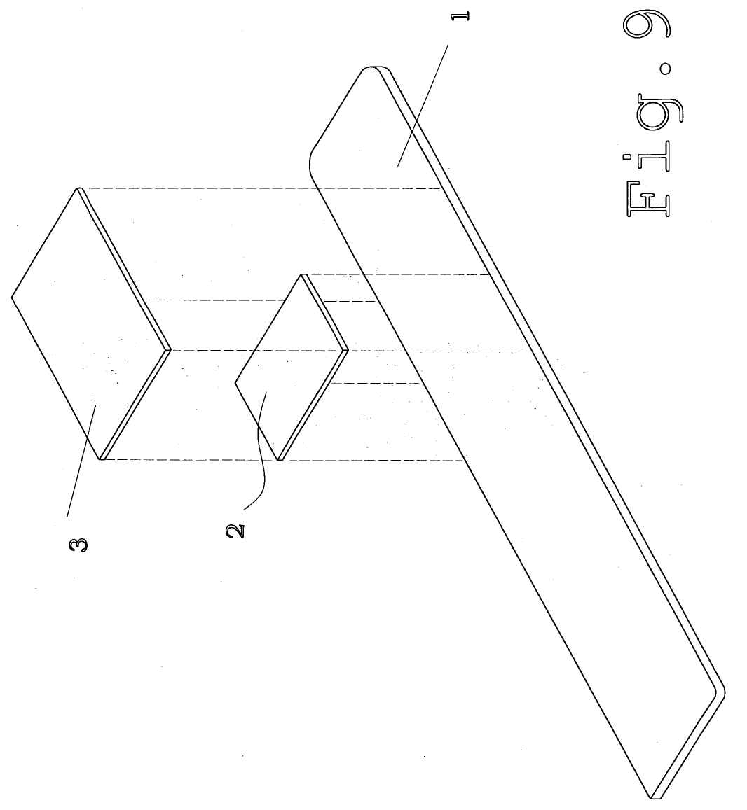

FIGS. 7 and 8 show a test strip according to the third embodiment of the present invention. According to this embodiment, the test strip comprises a substrate 1, a first sample-collection pad 2 adhered to one side of the substrate 1, and a second sample-collection pad 3 adhered to the outer surface of the first sample-collection pad 2. The side notch 21 of the first sample-collection pad 2 according to this embodiment has a rectangular shape. The second sample-collection pad 3 is relatively smaller than the first sample-collection pad 2, and adhered to a part of the outer surface of the second sample-collection pad 2. When the second sample-collection pad 3 adhered to the first sample-collection pad 1, the second sample-collection pad 3 and the first sample-collection pad 2 define an entrance 4 accessible to the blood sample receiving chamber (the side notch 21 of the first sample-collection pad 2). This embodiment can be used to examine a particular item of the patient blood in same manner as the aforesaid first and second embodiments.

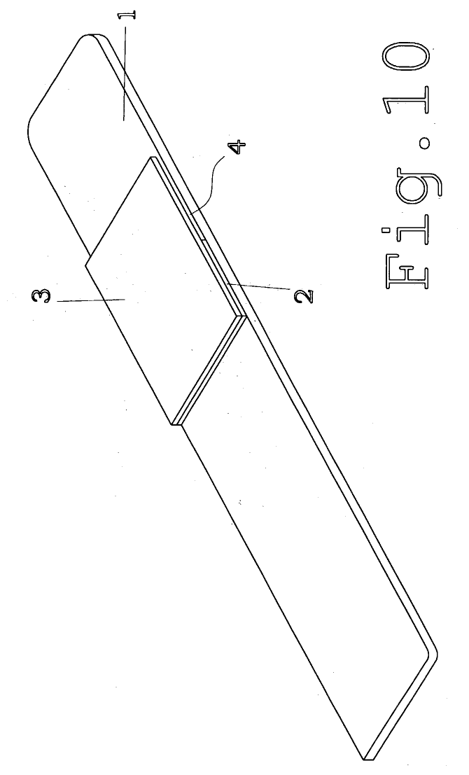

FIGS. 9 and 10 show a test strip according to the fourth embodiment of the present invention. According to this embodiment, the test strip comprises a substrate 1, a first sample-collection pad 2 adhered to one side of the substrate 1, and a second sample-collection pad 3 adhered to the first sample-collection pad 2. The second sample-collection pad 3 is relatively greater than the first sample-collection pad 2. When assembled, the second sample-collection pad 3 and the first sample-collection pad 2 define with the substrate 1 a blood sample receiving chamber having an entrance 4 in three sides.

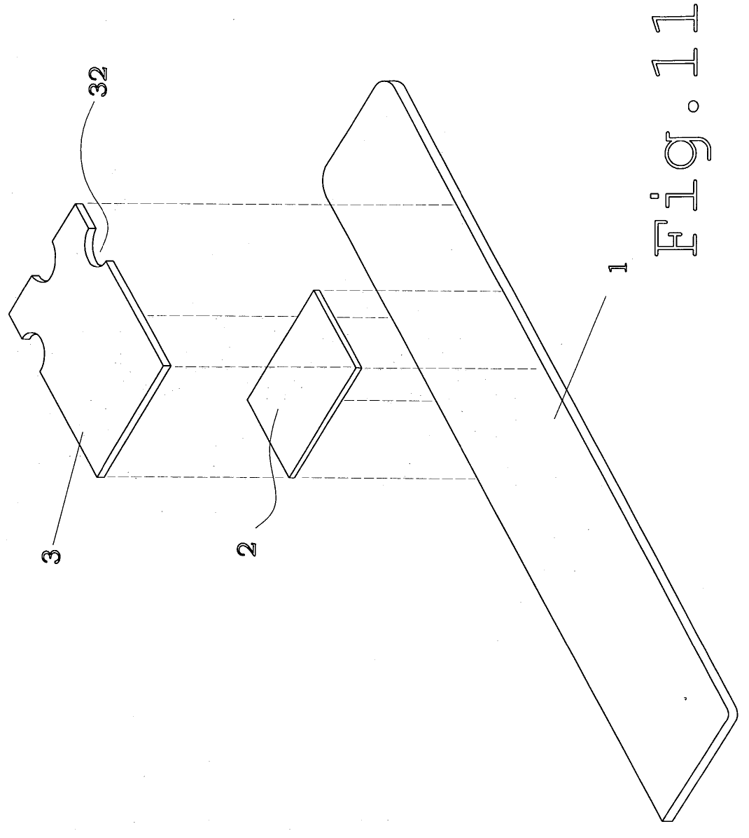

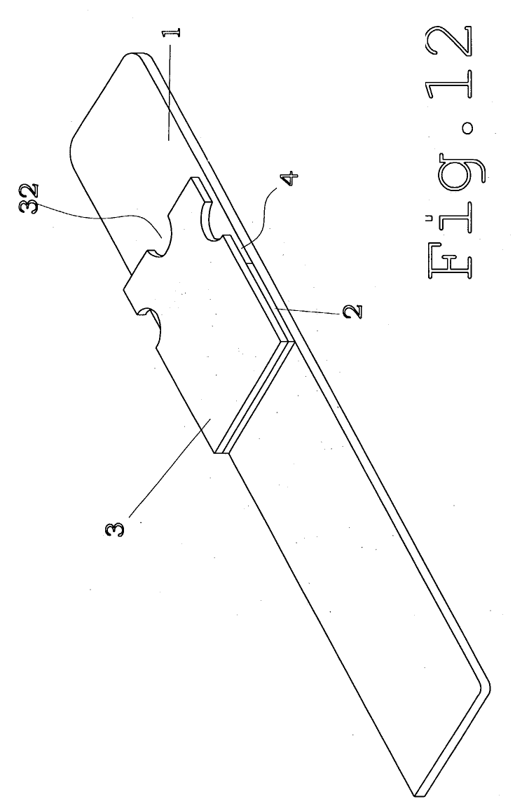

FIGS. 11 and 12 show a test strip according to the fifth embodiment of the present invention. This embodiment is similar to the aforesaid fourth embodiment with the exception of the shape of the second sample-collection pad 3. The second-sample collection pad 3 has notches 32 in three of the four sides thereof. When the test strip assembled, the notches 32 are disposed in communication with the blood sample receiving chamber defined by the sample-collection pads 2 and 3 and the substrate 1.

A prototype of test strip has been constructed with the features of FIGS. 1˜12. The test strip functions smoothly to provide all of the features discussed earlier.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A test strip comprising
a substrate;
a first sample-collection pad adhered to one side of said substrate, said first sample-collection pad having a side notch forming with said substrate a blood sample receiving chamber; and
a second sample-collection pad adhered to an outer surface of said first sample-collection pad in parallel to said substrate and defining a blood sample entrance accessible to said blood sample receiving chamber.

2. The test strip as claimed in claim 1, wherein said second sample-collection pad has an elongated slot extended to one side thereof and forming said blood sample entrance when said second sample-collection pad adhered to said first sample-collection pad.

3. The test strip as claimed in claim 1, wherein said second sample-collection pad is comprised of two parallel parts adhered to said first sample-collection pad in parallel with a narrow gap defined therebetween, forming said blood sample entrance.

4. A test strip comprising:
a substrate;
a first sample-collection pad adhered to one side of said substrate; and
a second sample-collection pad adhered to an outer surface of said first sample-collection pad in parallel to said substrate, said second sample-collection pad having an area greater than said first sample-collection pad such that said second sample-collection pad and said first sample-collection pad defining with said substrate a blood sample receiving open chamber having a blood sample entrance in three directions.

5. The test strip as claimed in claim 4, wherein said second sample-collection pad has at least one peripheral notch.
